(19) United States
(12) Patent Application Publication
(10) Pub. No.: US 2010/0106069 A1
(43) Pub. Date: Apr. 29, 2010

(54) WOUND COMPRESS AND METHODS

(76) Inventor: Jose Arias, Miami, FL (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/650,204

(22) Filed: Dec. 30, 2009

Related U.S. Application Data
(62) Division of application No. 11/751,948, filed on May 22, 2007, now abandoned.
(60) Provisional application No. 60/803,188, filed on May 25, 2006.

(51) Int. Cl. A61L 15/44; A61F 13/00

(52) U.S. Cl. 602/43; 602/48

(57) ABSTRACT

A method of treating a wound may include introducing a wound compress into the wound and compressing it into the wound. The wound compress may include a sealed, fenestrated pouch, and particles inside the pouch, each particle having a smallest linear dimension larger than the largest linear dimension of the pouch fenestrations.

### CROSS-REFERENCE TO RELATED APPLICATION

This application is a division of U.S. patent application Ser. No. 11/751,948, filed May 22, 2007, which claims the benefit of U.S. provisional application Ser. No. 60/803,188, filed May 25, 2006, both of which applications are hereby incorporated herein by reference.

### SUMMARY

The present disclosure describes wound compresses, kits including wound compresses, methods of making wound compresses, and methods of using wound compresses.

In an embodiment, a wound compress may include a sealed, fluid permeable, fenestrated pouch, and a quantity of particles inside the pouch, each particles having a smallest linear dimension larger than the largest linear dimension of the pouch fenestrations.

### BRIEF DESCRIPTION OF THE DRAWINGS

FIG. 1 is a schematic cross-sectional view of a compress disposed in a wound and covered with an overwrap.

FIG. 2 is a schematic view of a compress.

FIG. 3 is a schematic view of a compress having a fenestrated pouch.

FIG. 4 is a schematic view of an exemplary use of a compress.

FIG. 5 is a schematic view of an exemplary use of a compress and an overwrap.

FIG. 6 is a photograph of an exemplary embodiment of a wound compress.

FIG. 7 depicts detail of the wound compress embodiment shown in FIG. 6.

FIG. 8 is a photograph of an exemplary embodiment of a wound compress pressed against tissue.

FIG. 9 is a photograph of a clay mold wound simulation against which an exemplary wound compress embodiment was pressed.

FIG. 10 depicts detail from FIG. 9.

FIG. 11 is a photograph of a kit that includes a compress and an overwrap.

### DETAILED DESCRIPTION

Body tissue injuries often involve bleeding, and control of such bleeding may be an important part of initial treatment. Exsanguination from a wound is a significant source of morbidity and mortality, especially when the wound is severe. Control of bleeding within the first minutes or even seconds of a subject's receiving a wound may be essential for the subject's survival.

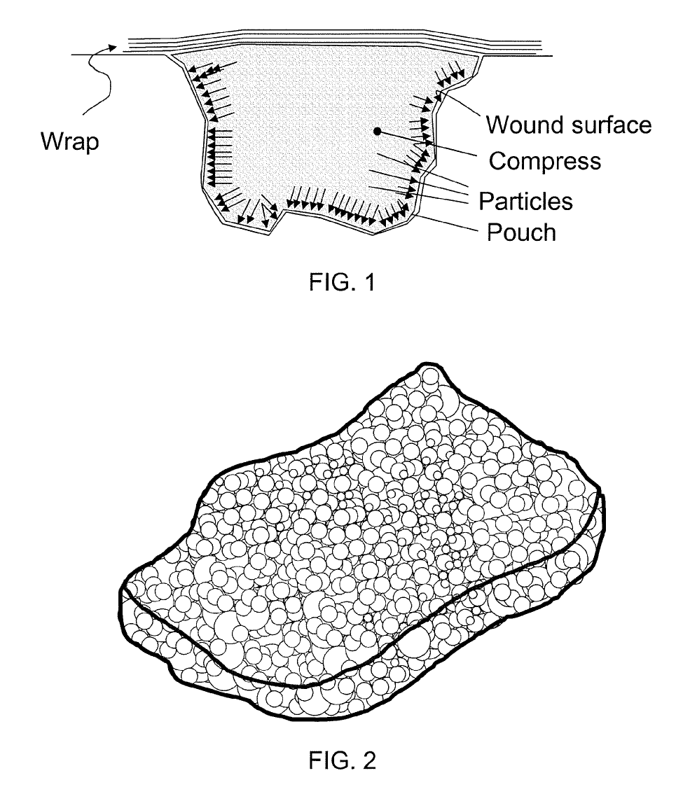

Bleeding control is improved by distributing the compressive force imparted by a compress to a wound surface over as much of the wound surface as possible and/or by increasing the amount of surface-area contact between the wound and the wound dressing or compress. In the case of a wound that removes tissue from a subject and leaves a cavity, the wound surface may include the surface area of the cavity, as depicted in FIG. 1. One way to accomplish one or both of these is to make the compress so flexible as to conform to the wound against which it is placed. Another approach is to fill the compress with a particulate or otherwise discretized material that can be shaped to conform to a wound.

These two approaches may be combined by forming a wound compress from a flexible pouch containing a quantity of particles, such as depicted in FIGS. 1-3. Once the compress is placed in a wound, an overwrap may be wrapped around it to help press the compress into the wound, secure the compress, and/or protect the wound from further injury or infection. Because the compress can conform closely to the wound contour, its compressive force upon the wound surface (represented by arrows along the periphery of the compress) is distributed over the surface area of contact between the compress and the wound. Applying compression to all or a majority of damaged blood vessels or other tissue leaking blood may create a tamponade effect and stanch the bleeding.

The pouch may be permeable or impermeable to liquid or air, but it should be impermeable to the particles. Put another way, if the pouch has holes, the holes should be small enough so that the particles cannot fall out of the pouch.

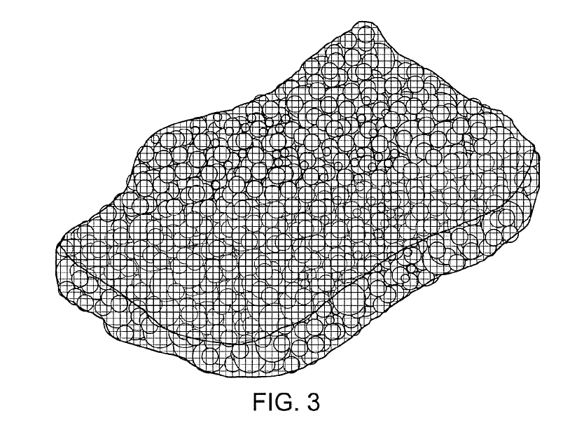

The pouch can be made from a wide variety of flexible materials, including fabric, mesh, netting, plastic, and/or a variety of polymers. The pouch may form a closed covering, such as if it is made with fabric, or a fenestrated opening, such as if it is made from mesh or netting. In some embodiments, the pouch is formed from a sparse netting that occupies less than 50%, 40%, 30%, 25%, 20%, 15%, 10%, 9%, 8%, 7%, 6%, 5%, 4%, 3%, 2%, or 1% of the surface area of the pouch (that is, the gaps between the strands of netting are larger than the strands themselves). A sparse netting allows the contents of the pouch to nestle against and closely conform to a wound surface. An example of a pouch with a sparse netting is depicted in FIG. 3. The fenestrations may have a largest linear dimension of no more than 1 centimeter, 50 millimeters, about 25 millimeters, about 20 millimeters, about 10 millimeters, about 5 millimeters, about 3 millimeters, about 2 millimeters, and/or about 1 millimeter.

The pouch may be filled, completely or partially, with a particulate material. The particulate nature of the filler allows it to conform to a surface against which it is pressed. The particles may be of uniform size or may have a range of sizes. The particles may be sufficiently large that they cannot escape the pouch through holes or other openings in the pouch. For example, if a pouch defines fenestrations having a largest linear dimension of 3 millimeters, then the smallest linear dimension of the particles should exceed 3 millimeters. In some embodiments, the smallest linear dimension of the particles exceeds the largest linear diameter of the fenestrations by 10%, 20%, 30%, 40%, or 50% of one or the other dimension, as a safety measure to help ensure that particles remain inside the pouch.

The particles made be made using a wide variety of materials, including polymers, plastics, and various natural materials. In some embodiments, the particles are made with polystyrene, such as foam polystyrene, polystyrene beads, expanded polystyrene beads, and/or extruded polystyrene. The material may be so compressible as to deform against a wound surface in response to pressure applied by a user's hand or by an overwrap. The material may be so rigid as to resist deformation under such pressure. The material may be so selected as to minimize the mass or weight of the compress, so selected as to maximize the compressive effect, or so selected as to balance weight and effect. For example, expanded polystyrene beads are both lightweight and compressible by hand pressure or an overwrap. The material may be biodegradable or bionondegradable. It may be fluid- and/or gas-permeable or impermeable. It may be fluid absorbent or nonabsorbent.

The particles may include a wide variety of natural materials, such as grains, nuts, seeds, shells, hulls, etc. For example, particles can be grains (or pieces thereof) of rice, barley, and/or other grains, hulled or unhulled. Grain hulls, such as buckwheat hulls, may be used. Shells, crushed or uncrushed, may be used; one example is crushed walnut shells.

Particles may be formed with an active substance, such as a hemostatic agent, a blood coagulant, an anesthetic, an antiseptic, and/or an antibiotic. The particles may be integrally formed with or coated with an active substance. The active substance may be provided as pellets mixed with the particles. Examples of hemostatic agents include zeolite (as described in U.S. Pat. No. 4,822,349, hereby incorporated herein by reference) and chitosan (natural or synthetic).

Particles may have a wide variety of shapes, including bead, spherical, spheroid, other curved shapes, polyhedron, irregular, and/or random shapes. Curved shapes may facilitate molding the compress to conform to a wound.

An overwrap may be provided with the compress. The overwrap may be a strip or band of elastic material, such as latex rubber. The overwrap may have a length sufficient to permit its being wrapped at least once, and preferably more than once, around the body part receiving the wound. A body part may be, for example, an appendage, the torso, an/or the head. Exemplary resting lengths may range from about 50 centimeters to about 200 centimeters, although other lengths may be used. The overwrap may have a width large enough so that it can completely cover the compress when wrapped over the compress (as shown in FIG. 1). Such complete covering of a wound may be helpful or necessary to prevent further injury, help avoid infection, or enable the subject to travel through water, mud, or other terrain from which the wound should be isolated. Alternatively, the overwrap may be so narrow as not to cover the compress completely. The overwrap may be stretched tightly as it is wrapped around a body part to help increase the force exerted on the wound surface by the compress. The overwrap may be wrapped around the body part until all or nearly of its length is wrapped around the body part. The free end of the wrap may be tucked under one or more layers of the wrap or be secured to the wrap using, e.g., a clasp, hook, adhesive, and/or hook and loop fasteners, among others.

A compress may be provided in a sealed container, such as an airtight, vacuum-sealed, and/or sterilized container. A compress may be provided together with an overwrap in such a container.

Each material in a compress, overwrap, container, and/or kit may be sterile or sterilizable. The materials may be sterile before assembly into a compress, overwrap, container, and/or kit, sterilized during assembly, sterilized after assembly, or sterilized at more than one point before, during, and/or after assembly.

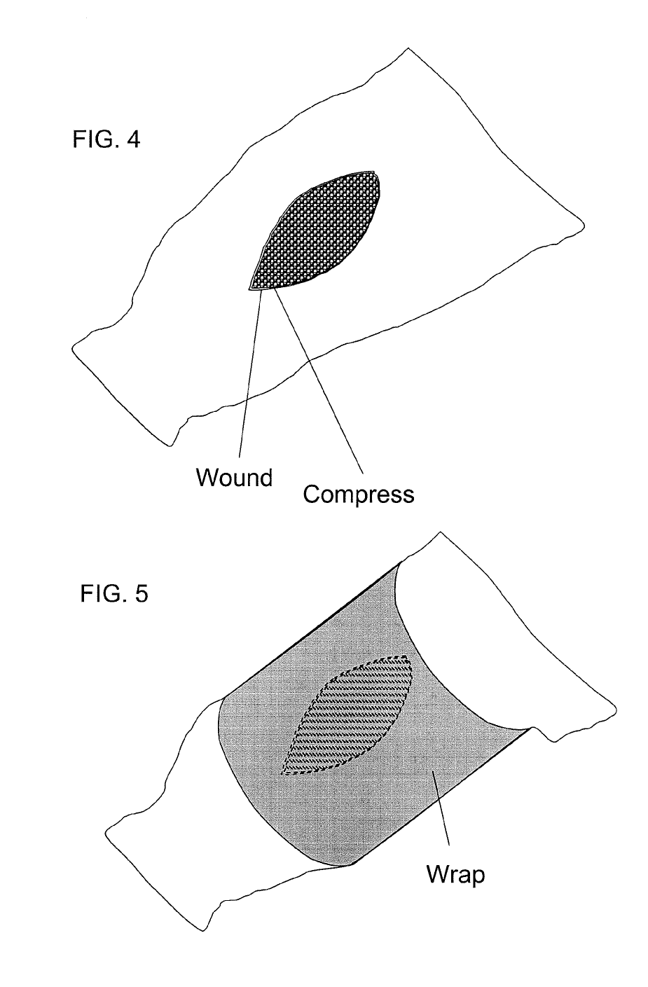

In use, a compress may be snugly fitted into a wound (FIG. 4) so that it conforms to the wound surface. An overwrap may be tightly wound over the wound and compress and around the affected body part (FIG. 5) to provide compressive force and/or sealing. In some cases, the overwrap may be so tightly wrapped as to act as a tourniquet.

A second compress (or more) may be positioned so that it is also compressed by the overwrap. For example, a second compress may be positioned on the opposite side of the body part from where the wound and first compress are positioned. The overwrap may then be wrapped over the wound and both compresses. A second compress may cushion unwounded tissue from the overwrap's compressive force and lessen or prevent discomfort.

One or more wound compresses disclosed herein may be combined with one or more tourniquets disclosed in U.S. Provisional Application No. 60/805,391, filed Jun. 21, 2006, which is hereby incorporated herein by reference. A compress and a tourniquet may be combined into a single package or kit, optionally along with accessories. For example, a wound compress and elastic band may be packaged with a tourniquet. Such packages or kits may be issued, for example, to military personnel in combat situations. An elastic band may be included in the package or kit that is usable both a covering for a wound compress and as the elastic band for a tourniquet.

### Example

The present description is further illustrated by the following example, which should not be construed as limiting the claims in any way.

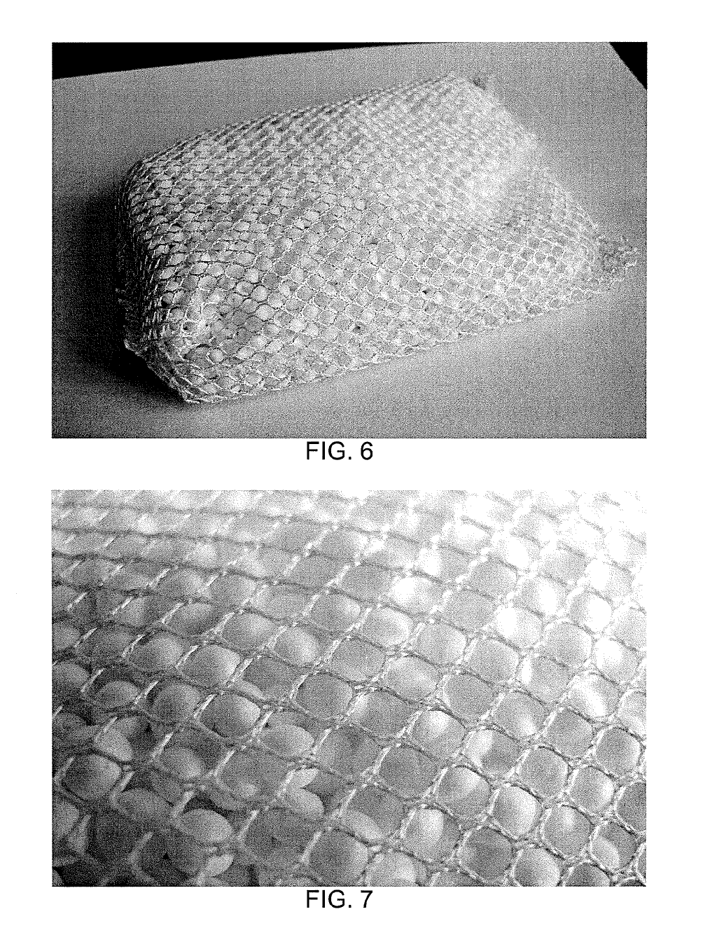

FIG. 6 depicts an exemplary embodiment of a wound compress. The compress includes a sealed, fenestrated, flexible pouch that holds a quantity of particles sufficiently large that they cannot leave the pouch through the fenestrations. FIG. 7 shows detail of the compress depicted in FIG. 1. In this exemplary embodiment, the pouch is formed from cotton netting having fenestrations of about 3 millimeters wide. The particles are expanded polystyrene beads of varying sizes larger than the fenestrations.

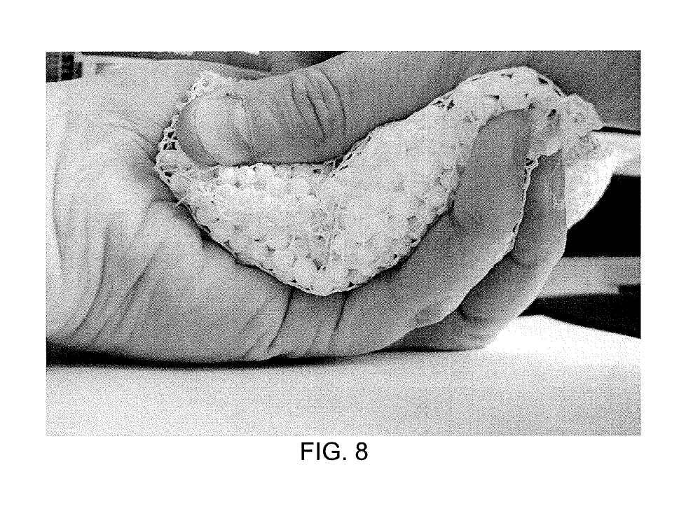

FIG. 8 depicts a simulated exemplary use of a wound compress. The flexible nature of the container allows the compress to conform to the tissue against which it is pressed. The effect is illustrated by using the bottom hand to simulate a wound cavity and the top hand to simulate an overwrap or other compression source. In actual use, the compress would be pressed against wounded tissue.

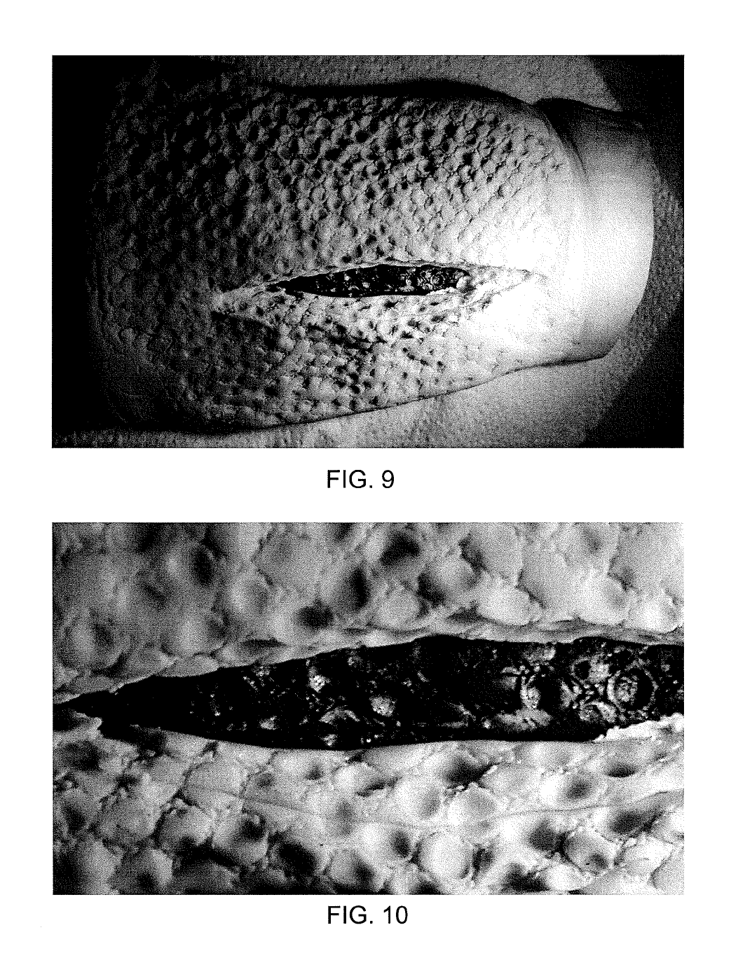

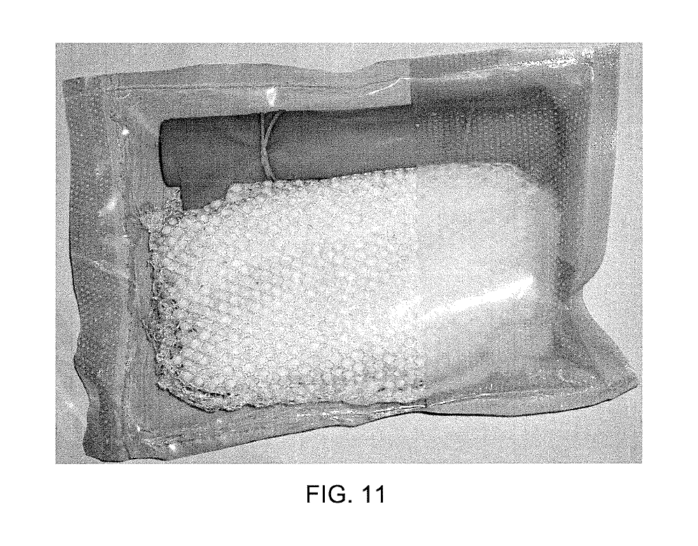

FIGS. 9-10 further illustrate the ability of the compress to conform to the tissue against which it is placed and distribute force over a wound surface. A simulated laceration was created in a piece of modeling clay to resemble a typical wound that might be treated with a wound compress. A compress was pressed against the simulated wound, and the particles in the compress left impressions in the modeling clay. FIG. 9 and FIG. 10 (closeup of a portion of FIG. 9) shows that the particles contacted the entire wound surface. The particle impressions have roughly the same depth, which suggests that the compressive force was distributed over the wound surface roughly uniformly.

The compress and an overwrap, a rubber latex resistance band typically used for exercise and rehabilitation, are provided together in a sealed package (FIG. 11). The package is vacuum sealed to reduce its volume and lessen the risk of accidental opening.

I claim:

1. A wound compress comprising:
a sealed, fluid permeable, fenestrated pouch; and
a quantity of particles inside the pouch, each particles having a smallest linear dimension larger than the largest linear dimension of the pouch fenestrations.

2. The wound compress of claim 1, wherein the pouch is formed from netting.

3. The wound compress of any preceding claim, wherein the pouch fenestrations have a largest linear dimension of no more than about 3 millimeters.

4. The wound compress of claim 1, wherein the particles comprise polystyrene.

5. The wound compress of claim 4, wherein the particles comprise expanded polystyrene beads.

6. The wound compress of claim 1, further comprising an active ingredient.

7. The wound compress of claim 6, wherein the active ingredient comprises at least one of a hemostatic agent, a blood coagulant, an anesthetic, an antiseptic, and an antibiotic.

8. The wound compress of claim 6, wherein the active ingredient comprises chitosan.

9. The wound compress of claim 6, wherein the active ingredient comprises zeolite.

10. The wound compress of claim 6, wherein the active ingredient is coated on one or more particles.

11. The wound compress of claim 6, wherein at least one particle is integrally formed with the active ingredient.

12. The wound compress of claim 6, wherein the active ingredient is provided as pellets mixed with the particles.

13. A wound compress comprising:
a sealed and fluid permeable pouch formed from fabric netting and having fenestrations having a largest linear dimension of no more than about 3 millimeters; and
a quantity of expanded polystyrene beads inside the pouch, each particle having a smallest linear dimension larger than the largest linear dimension of the pouch fenestrations.

14. A kit comprising a wound compress defined by claim 1 and an overwrap.

15. The kit of claim 14, wherein the overwrap comprises a band of elastic material.

16. The kit of claim 15, wherein the elastic material comprises latex rubber.

17. A method of treating a wound in a body part of a subject comprising:
introducing a wound compress into a cavity left by the wound, the cavity having a surface area, the compress being sufficiently flexible as to allow it to conform closely to the wound cavity, and the compress comprising:
a sealed, fenestrated pouch; and
particulate material inside the pouch, the particulate material sized and shaped to allow the compress to be shaped to conform to the wound cavity, each particle of the particulate material having a smallest linear dimension larger than the largest linear dimension of the pouch fenestrations; and
compressing the wound compress into the wound cavity so that the compress conforms closely to the cavity's surface area, exerts compressive force distributed over the wound surface area, and stanches bleeding from the wound.

18. The method of claim 17, further comprising covering the wound and the wound compress.

19. The method of claim 18, wherein the wound and the wound compress are covered with an overwrap.

20. The method of claim 19, further comprising wrapping the overwrap around the body part.
